(19) United States
(12) Patent Application Publication
(10) Pub. No.: US 2024/0416036 A1
(43) Pub. Date: Dec. 19, 2024

(54) DISPOSABLE CASSETTE FRONT MODULE

(71) Applicant: SHL Medical AG, Zug (CH)

(72) Inventor: Ming-Ting Yin, Taoyuan City (TW)

(21) Appl. No.: 18/702,948

(22) PCT Filed: Oct. 7, 2022
(86) PCT No.: PCT/EP2022/077946

§ 371 Date: Apr. 19, 2024

(30) Foreign Application Priority Data
Oct. 22, 2021 (EP) 21204331.9

(51) Int. Cl. A61M 5/20

(52) U.S. Cl. CPC A61M 5/20

(57) ABSTRACT

The present disclosure provides a disposable cassette front module for a medicament delivery device. The medicament delivery device includes a reusable rear module which forms a rear portion of the medicament delivery device and to which the cassette front module is configured to be detachably connected to form a front portion of the medicament delivery device. The disposable cassette front module includes an outer cover configured to accommodate a syringe, the outer cover having a proximal outer cover end and a distal outer cover end. The disposable cassette front module further includes a power pack assembly comprising a plunger rod and a drive device configured to drive the plunger rod from an initial axial position relative to the outer cover towards the proximal outer cover end.

### CROSS-REFERENCE TO RELATED APPLICATIONS

The present application is a U.S. National Phase Application pursuant to 35 U.S.C. § 371 of International Application No. PCT/EP2022/077946 filed Oct. 7, 2022, which claims priority to European Patent Application No. 21204331.9 filed Oct. 22, 2021. The entire disclosure contents of these applications are herewith incorporated by reference into the present application.

### TECHNICAL FIELD

The present disclosure generally relates to medicament delivery devices.

### BACKGROUND

Some medicament delivery devices are configured to be reused. Thus, when the device has been emptied, a new syringe or medicament cartridge may be placed in the device. In case the medicament delivery device is an autoinjector, the device may be reloaded.

An example of a reloadable autoinjector is disclosed in WO9632974 A1. The auto-injector comprises a housing portion designed to receive a syringe and a cover portion. The auto-injector includes a re-cocking means for re-cocking the auto-injector.

### SUMMARY

It is desirable, for example from an environmental and cost perspective, to provide reusable medicament delivery devices. Some users may however have an impaired physical capability to for example re-cock a reloadable autoinjector.

An object of the present disclosure is thus to provide a disposable cassette front module and a medicament delivery device comprising the disposable cassette front module which solve, or at least mitigate, problems of the prior art.

There is hence according to a first aspect of the present disclosure provided a disposable cassette front module for a medicament delivery device comprising a reusable rear module which forms a rear portion of the medicament delivery device and to which the cassette front module is configured to be detachably connected to form a front portion of the medicament delivery device, the cassette front module comprising: an outer cover configured to accommodate a syringe, the outer cover having a proximal outer cover end and a distal outer cover end, and a power pack assembly comprising a plunger rod and a drive device configured to drive the plunger rod from an initial axial position relative to the outer cover towards the proximal outer cover end.

The disposable cassette front module thus forms a disposable part of a medicament delivery device also comprising a reusable rear module. The disposable cassette front module in particular comprises the power pack assembly, including the plunger rod and a drive device for driving the plunged rod. Thus, since the cassette front module is disposable, the user does not have to reload the cassette front module before medicament administration. This makes the medicament delivery device more user friendly.

Furthermore, more flexibility is provided for the designer to adjust or change the drive device depending on the viscosity or concentration of the medicament. For example, in case the drive device is a drive spring, the drive spring can have different spring constant in different cassette front modules.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

The drive device may for example be a drive spring or it may comprise compressed gas or expandable polymers for driving the plunger rod.

According to one embodiment the drive device is a drive spring, and wherein the cassette front module comprises a first sleeve member arranged around the plunger rod and configured to engage with the plunger rod to hold the plunger rod in the initial axial position.

According to one embodiment the first sleeve member has a distal end provided with a proximal radial surface and the drive spring has a distal drive spring end bearing against the proximal radial surface.

According to one embodiment the plunger rod has a hollow interior configured to receive the drive spring, wherein the plunger rod has a distal radial surface, and the drive spring has a proximal drive spring end bearing against the distal radial surface.

According to one embodiment the plunger rod has plunger rod recesses and the first sleeve member has radially flexible arms configured to engage with a respective one of the plunger rod recesses to hold the plunger rod in the initial axial position, wherein the cassette front module comprises a second sleeve member arranged concentrically with and radially outside the first sleeve member, the second sleeve member having an inner surface with sleeve recesses configured to receive a respective one of the radially flexible arms when the second sleeve member is in a release rotational position relative to the plunger rod, wherein the second sleeve member is configured to be rotationally locked relative to the first sleeve member in an initial rotational position to prevent the first sleeve recesses to move to the release rotational position. Rotation of the second sleeve member is thus prevented before the cassette front module has been connected to the rear module.

According to one embodiment the second sleeve member comprises a radially flexible circumferentially extending arm provided with a hook and the first sleeve member has a radial opening configured to receive the hook to prevent the second sleeve member to rotate in a first direction but to enable the second sleeve member to rotate in a second direction.

According to one embodiment the second sleeve member comprises a radially flexible axial tab extending radially outwards with an increasing amount in an axial direction towards the proximal outer cover end, and the first sleeve member has a stop extending axially towards the distal outer cover end adjacent to the tab, the stop being configured to prevent the second sleeve member to rotate in a second direction.

The radially flexible circumferentially extending arm with its hook and the radial opening thus prevents rotation of the second sleeve member in the first direction before the cassette front module has been connected to the rear module. The tab and stop prevents rotation of the second sleeve member in the second direction before the cassette front module has been connected to the rear module.

According to one embodiment the outer cover has an inner surface provided with an axial rib arranged axially and radially aligned with the tab, the axial rib having a distal end face closer to the proximal outer cover end than the tab, the tab thereby restricting axial movement of the outer cover relative to the second sleeve member in a direction towards the tab.

Linear movement of the outer cover in the distal direction is thus restricted before the cassette front module has been connected to the rear module.

One embodiment comprises a cap configured to be attached to the outer cover, wherein the proximal outer cover end has a proximal opening and the cap comprises flexible axial arms configured to extend into the outer cover through the proximal opening, wherein the outer cover has an inner surface provided with radially inwards extending protrusions configured to bear against a respective one of the flexible axial arms at an axial position of the outer cover where, in use, a syringe is arranged, the protrusions being configured to prevent the flexible axial arms to move in an axial direction towards the proximal outer cover end and the syringe restricting radial inwards flexing of the flexible axial arms.

The syringe is arranged radially inwards of the flexible arms and radially inwards extending protrusions and fills out radial space radially inwards of the flexible arms so that they cannot flex radially inwards with such as amount that they can disengage from the radially inwards extending protrusions.

There is according to a second aspect provided a medicament delivery device comprising: a reusable rear module comprising a housing having a proximal housing end and a distal housing end, the proximal housing end having a proximal housing opening, and the cassette front module as claimed in any of the preceding claims, wherein the outer cover is configured to be received by the proximal housing opening, the cassette front module being configured to be moved from an extended position relative to the housing to a retracted position, wherein the rear module comprises a resilient member configured to bias the cassette front module towards the extended position.

According to one embodiment the rear module comprises: an interface sleeve biased towards the proximal housing end, the interface sleeve bearing against a distal edge of the outer cover, wherein the outer cover has an external axial rib, and the interface sleeve comprises an axial recess configured to engage with the external axial rib to prevent rotation of the interface sleeve relative to the outer cover, and a rotator provided with a guide structure, the rotator being rotationally engaged with the second sleeve member and being configured to press the tab radially inwards to enable rotation of the second sleeve member in the second direction, wherein the interface sleeve is configured to cooperate with the guide structure when the cassette front module is moved from the extended position towards the retracted position causing the rotator and the second sleeve member to rotate in the second direction.

According to one embodiment the rear module comprises a release switch sleeve which is torsionally biased and axially locked relative to the housing, the release switch sleeve comprising a plurality of anchoring hooks and the first sleeve member comprises a plurality of protrusions configured to engage with a respective one of the anchoring hooks to prevent the rear module from disengaging from the cassette front module.

According to one embodiment the release switch sleeve comprises a release switch extending through a circumferentially extending slot of the housing, wherein the release switch is configured to be slid in the slot from a first position towards which it is torsionally biased to a second position to disengage the anchoring hooks from the protrusions and thereby disengage the rear module from the cassette front module.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows a perspective view of an example of a partly reusable medicament delivery device before full assembly;

FIG. 2 shows the medicament delivery device in FIG. 1 after it has been assembled;

FIG. 3 is an exploded view of an example of a cassette front module;

FIG. 4 is a side view of a front portion of the cassette front module before assembly with a rear module;

FIG. 5 shows the cap being pulled of the cassette front module after assembly with a rear module;

FIG. 6 shows a perspective view of a rear portion of the cassette front module;

FIG. 7 depicts examples of a first and second sleeve member;

FIG. 8 is an exploded view of an example of a rear module;

FIG. 9 depicts the medicament delivery device in an assembled state with a housing removed;

FIG. 10 shows a close-up view of a connection between the cassette front module and the rear module;

FIG. 11 shows a detail of a rotator;

FIGS. 12 and 13 show the medicament delivery device prior to medicament injection;

FIG. 14 shows the medicament delivery device during injection;

FIG. 15 depicts the medicament delivery device during injection without its housing;

FIG. 16 shows interaction between internal components during medicament injection;

FIG. 17 depicts the medicament delivery device after injection has been completed;

FIG. 18 shows the medicament delivery device when the rear module is set to be released from the cassette front module after injection;

FIG. 19 shows a close-up view of how the cassette front module is released from the rear module;

FIG. 20 shows the medicament delivery device after the disposable cassette front module has been disconnected from the reusable rear module; and

FIG. 21 depicts a locked-out state of the cassette front module after it has been disconnected from the rear module.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

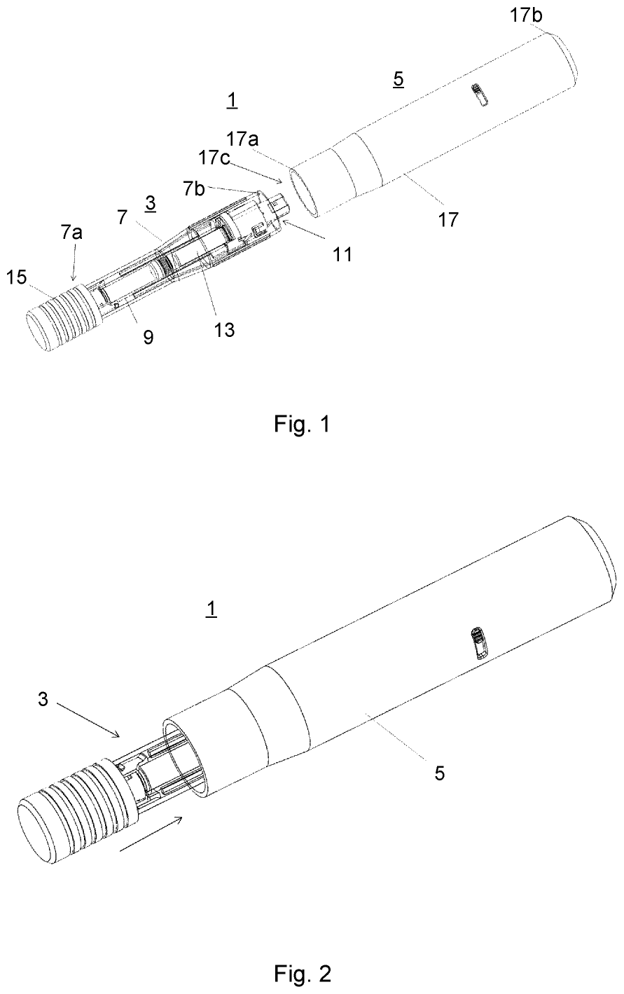

FIG. 1 shows a perspective view of a partially reusable medicament delivery device 1. The medicament delivery device 1 comprising a disposable cassette front module 3 and a reusable rear module 5.

The medicament delivery device 1 may be an autoinjector.

The cassette front module 3 is configured to be detachably connected to the rear module 5. In FIG. 1, the medicament delivery device 1 is depicted before the cassette front module 3 and the rear module 5 have been connected to each other.

The cassette front module 3 forms a front portion of the medicament delivery device 1 when connected to the rear module 5. The rear module 5 forms a rear portion of the medicament delivery device 1.

The cassette front module 3 has an outer cover 7. The outer cover 7 has a proximal outer cover end 7a and a distal outer cover end 7b.

The outer cover 7 has a through-opening extending from the proximal outer cover end to the distal outer cover end 7b. The outer cover 7 has a generally elongated shape as it extends from the proximal outer cover end 7a to the distal outer cover end 7b.

The cassette front module 3 is configured to accommodate a syringe or medicament container 9 inside the outer cover 7.

The cassette front module 3 comprises a power pack assembly 11. The power pack assembly 11 is when triggered configured to automatically discharge medicament from the syringe or medicament container 9.

The power pack assembly 11 includes a plunger rod 13 configured to move axially inside the outer cover 7 from an initial axial position towards the proximal outer cover end 7a, and a drive device (not shown) configured to drive the plunger rod from the initial axial position towards the proximal outer cover end 7a.

The drive device is in this example a drive spring arranged inside the plunger rod 13 which is hollow.

The cassette front module 3 comprises a cap 15 configured to be attached to the outer cover 7. The cap 15 is in the example configured to be fitted to the outer cover 7 at the proximal outer cover end 7a.

The rear module 5 comprises a housing 17 having a proximal housing end 17a and a distal housing end 17b. The proximal housing end 17a has a proximal housing opening 17c.

FIG. 2 shows when the cassette front module 3 and the rear module 5 have been connected to each other. The connection of the cassette front module 3 and the rear module 5 is made by inserting a distal end portion of the cassette front module 3 into the rear module 5 via the proximal housing opening 17c.

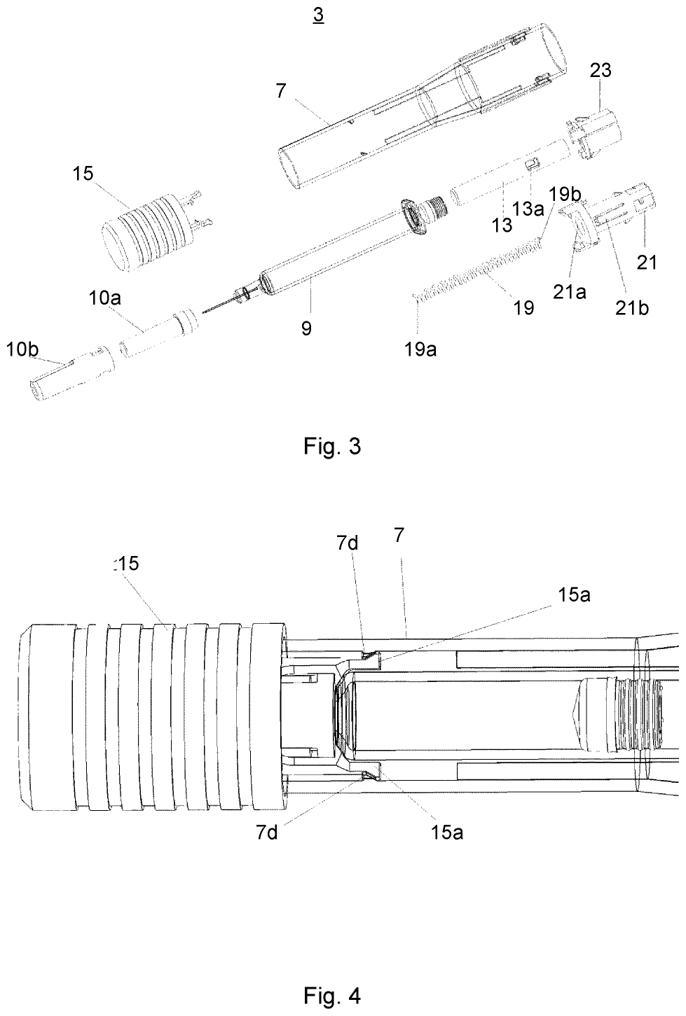

FIG. 3 shows an exploded view of the cassette front module 3. The cassette front module 3 comprises a first sleeve member 21 configured to be arranged around the plunger rod 13. The first sleeve member 21 has a proximally opening axial channel 21a in which a portion of the plunger rod 13 is arranged.

The first sleeve member 21 is configured to engage with the plunger rod 13 to hold the plunger rod in the initial axial position. The plunger rod 13 has radial plunger rod recesses, such as plunger rod recess 13a, and the first sleeve member 21 has radially flexible arms, such as radially flexible arm 21b, configured to engage with a respective one of the plunger rod recesses 13a to hold the plunger rod 13 in the initial axial position.

The cassette front module 3 comprises a second sleeve member 23. The second sleeve member 23 is arranged concentrically with and radially outside the first sleeve member 21.

The second sleeve member 23 is configured to be rotated relative to the first sleeve member 21 when the cassette front module 3 has been connected to the rear module 5.

In an initial rotational position, the second sleeve member 23 is configured to prevent the radially flexible arms 21b to flex radially outwards. The first sleeve member 21 thus maintains its engagement with the plunger rod 13 as long as the second sleeve member is in the initial rotational position.

When the cassette front module 3 has been connected to the rear module 5 the second sleeve member 23 is able to be rotated relative to the first sleeve member 21 to obtain a release rotational position, in which the radially flexible arms 21b are allowed to flex radially outwards and disengage from the plunger rod 13.

The second sleeve member 23 is axially locked relative to the first sleeve member 21.

The cassette front module 3 comprises a drive device 19 configured to drive the plunger rod 13 from the initial axial position towards the proximal outer cover end 3a. The drive device 19 is in the following example a drive spring 19.

The drive spring 19 has a proximal drive spring end 19a and a distal drive spring end 19b.

The plunger rod 13 has a hollow interior which is configured to receive the drive spring 19.

The plunger rod 13 has a distal radial surface, i.e., a distally facing radial surface, arranged inside its hollow interior. The proximal drive spring end 19a bears against the distal radial surface.

The first sleeve member 21 has a proximal radial surface, i.e., a proximally facing radial surface, forming a distal end of the proximally opening axial channel 21a. The distal drive spring end 19b bears against the proximal radial surface.

The drive spring 19 is thus set in an energised state by compression between the proximal radial surface and the distal radial surface. The plunger rod 13 is set in a loaded state, in the initial axial position, independently of connection of the cassette front module 3 with the rear module 5. The plunger rod 13 is in a loaded state in the cassette front module 3 before the cassette front module 3 is connected to the rear module 5.

FIG. 4 depicts the cap 15 fitted to the outer cover 7 before the cassette front module 3 has been connected to the rear module 5. The cap 15 is designed so that it generally cannot be removed from the outer cover 7 before the cassette front module 3 has been connected to the rear module 5.

The cap 15 comprises flexible arms 15a configured to extend into the outer cover 7. The outer cover 7 has an inner surface provided with radially inwards extending protrusions 7d. The flexible arms 15a are configured to engage with the protrusions 7d when the cap 15 is fitted to the outer cover 7. The protrusions 7d are in this case closer to the proximal outer cover end 7a than the distal ends of the flexible arms 15a.

The flexible arms 15a are gradually increasing in radial thickness in a direction towards the distal outer cover end 7b. The gradually increasing radial thickness may form a wedge-shaped or ramp structure of the flexible arms 15a.

The protrusions 7d gradually increase the radial thickness of the outer cover 7 in a direction towards the proximal outer cover end 7a. The gradually increasing radial thickness may for example define a wedge-shaped or ramp structure formed by the protrusions 7d.

The two wedge-shaped structures face and may bear against each other when the cap 15 is arranged on the outer cover 7. Pulling the cap 15 away from the outer cover 7 results in that the two wedge-shaped structures are moved against each other. The syringe 9 is arranged radially inwards of the protrusions 7d and the flexible arms 15a before the cassette front module 3 has been connected to the rear module 5. The syringe 9 restricts radial inwards flexing of the flexible arms 15a when the cap 15 is being pulled from the outer cover 7. The cap 15 is thus kept from being pulled off the outer cover 7.

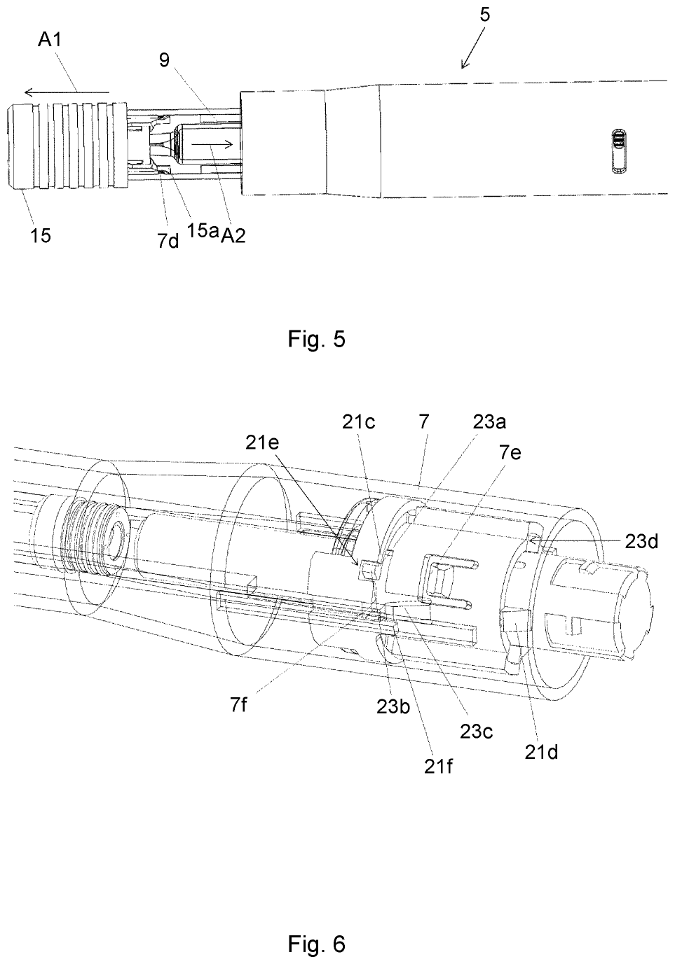

FIG. 5 shows a situation when the cassette rear module 3 has been connected to the rear module 5 and the cap 15 is being pulled off from the outer cover 7 in a direction shown by arrow A1.

There is a play between the internal components of the cassette front module 3 and the outer cover 7. When the cassette front module 3 has been connected to the rear module 5, the connection is made via the internal components. The connection of the rear module 5 is in particular made with the first sleeve member 21. This causes the internal components, including the syringe 9, to move in a direction from the proximal outer casing end 7a towards the distal outer casing end 7b as the connection is being made, shown by arrow A2. The syringe 9 has thus moved in the distal direction relative to the protrusions 7d when the medicament delivery device 1 has been assembled and before the cap 15 pulled off, leaving radial space for the flexible arms 15a to flex inwards. When the cap 15 is pulled off from the outer casing 7, the flexible arms 15a will therefore be able to flex radially inwards to disengage from, and axially move past the protrusions 7d.

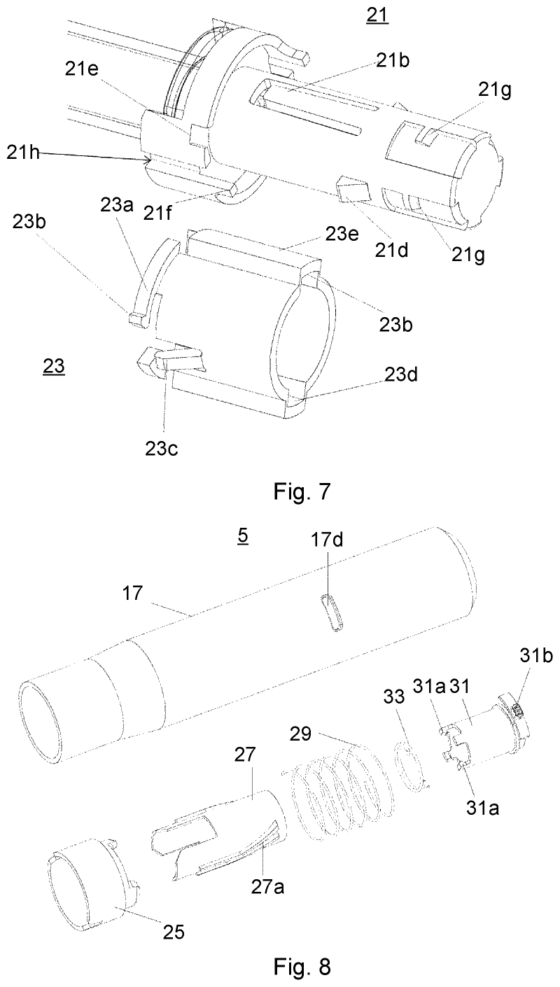

FIG. 7 shows a close-up view of a distal end portion of the cassette front module 3 before it is connected to the rear module 5. The internal components are in the example shown at their proximal end position. The internal components are able to move axially towards the distal outer cover end 7b until a distally facing radial flange surface 21c of the first sleeve member 21 reaches a radially inwards extending structure 7e of the outer cover 7.

The second sleeve member 23 comprises an inner surface provided with sleeve recesses 23d. The sleeve recesses 23d are arranged circumferentially offset from a respective flexible arm 21b of the first sleeve 23 when the second sleeve member 23 is in the initial rotational position relative to the first sleeve member 21. The sleeve recesses 23d are configured to align with and receive a respective one of the flexible arms 21b when the second sleeve member 23 has been rotated to obtain the release rotational position.

The second sleeve member 23 is rotationally locked relative to the first sleeve member 21 until the cassette front module 3 is connected to the rear module 5.

The first sleeve member 21 has a radial opening 21e. The radial opening 21e is in the present example provided in the flange surface 21c. The second sleeve member 23 comprises a radially flexible circumferentially extending arm 23a provided with a hook 23b configured to engage with the radial opening 21e when the second sleeve member 23 is in the initial rotational position. The radial opening 21e is configured to receive the hook 23b to engage the first sleeve member 21 with the second sleeve member 23. The hook 23b may have a wedge or ramp shape in the circumferential direction so that the hook 23b can disengage from the radial opening 21e when the second sleeve member 23 is rotated in one of two circumferential directions. This configuration thus prevents the second sleeve member 23 to rotate in a first direction relative to the first sleeve member 21 but enables the second sleeve member to rotate in a second direction opposite the first direction.

The first sleeve member 21 has a stop 21f extending axially towards the distal outer cover end 7b. The second sleeve member 23 has a radially flexible axial tab 23c extending radially outwards with an increasing amount in an axial direction towards the proximal outer cover end 7a. The stop 21f extends axially beyond and adjacent to the tab 23c and is arranged offset from the tab 23c in a circumferential direction of the outer cover 7. The stop 21f prevents the second sleeve member 23 to rotate in the second direction before the cassette front module 3 is connected to the rear module 5. The second sleeve member 21 is thus prevented from rotation in any direction as long as the cassette front module 3 is not connected to the rear module 5.

The outer cover 7 has an inner surface provided with an axial rib 7f. The axial rib 7f is axially and radially aligned with the tab 23c of the second sleeve member 23. Further, the first sleeve member 21 has an axial slot 21h in which the axial rib 7f can run and which is axially aligned with the tab 23c before the cassette front module 3 has been connected to the rear module 5. The axial rib 7f has a distal end face arranged closer to the proximal outer cover end 7a than the tab 23c. The tab 23c thus restricts axial movement of the outer cover relative to the second sleeve member 23 in a direction towards the tab 23c.

The first sleeve member 21 has a first axial stop structure 21d which prevents the second sleeve member 23 to move in a direction towards the distal outer cover end 7b relative to the first sleeve member 21. The distally facing radial flange surface 21c prevents the second sleeve member 23 to move in a direction towards the proximal outer cover end 7a relative to the first sleeve member 21. The second sleeve member 23 is thus axially locked relative to the first sleeve member 21 by means of the first axial stop structure 21d and the distally facing radial flange surface 21c.

FIG. 7 shows a detailed view of the first sleeve member 21 and the second sleeve member 23.

The first sleeve member 21 comprises a plurality of protrusions 21g extending radially outwards. The protrusions 21g are provided on a distal portion of the first sleeve member 21. The protrusions 21g have a gradually increasing radial dimension in a direction from the distal outer cover end 7b towards the proximal outer cover end 7a. The protrusions 21g may be wedge-shaped or shaped as a ramp. There is a planar surface in the circumferential direction adjacent to each protrusion 21g. The protrusions 21g are for connecting the cassette front module 3 to the rear module 5, as will be explained in more detail in the following.

The second sleeve member 23 has outer axial ribs 23e. The sleeve recesses 23d may be axially aligned with a respective one of the outer axial ribs 23e. Thus, the outer surface of the second sleeve member 23 may be provided with the outer axial ribs 23e and the inner surface may at a respective one of the these locations be provided with a sleeve recesses 23d.

FIG. 8 is an exploded view of the rear module 5. The rear module 5 comprises an interface sleeve 25, a rotator 27, and a resilient member 29 arranged in the housing 17.

The interface sleeve 25 is biased towards the proximal housing end 17a. The resilient member 29, in this example a spring, is configured to bias the interface sleeve 25 towards the proximal housing end 17a.

The rotator 27 has a sleeve structure arranged concentrically with the interface sleeve 25. The rotator 27 has a guide structure 27a provided on its outer surface. The guide structure 27a may comprise helically extending tracks.

The interface sleeve 25 comprises radially inwards extending pins configured to cooperate with the guide structure 27a. The pins are configured to run in the tracks.

The rear module 5 comprises a release switch sleeve 31 configured to be torsionally biased. The exemplified rear module 5 comprises a torsion spring 33 configured to torsionally bias the release switch sleeve 31. One end of the torsion spring 33 may for example be fixed to the housing 17 and the other end of the torsion spring 33 may be fixed to the release switch sleeve 31.

The release switch sleeve 31 is configured to be connected to the first sleeve member 21. The release switch sleeve 31 comprises a plurality of anchoring hooks 31a configured to engage with a respective one of the protrusions 21g of the first sleeve member 21.

The release switch sleeve 31 has a release switch 31b. The release switch 31b extends radially outwards. The housing 17 has a circumferentially extending slot 17d through which the release switch 31b extends. The release switch 31b is configured to be slid in the slot 17d from a first position towards which the release switch sleeve 31 and thus the release switch 31b is biased, to a second position.

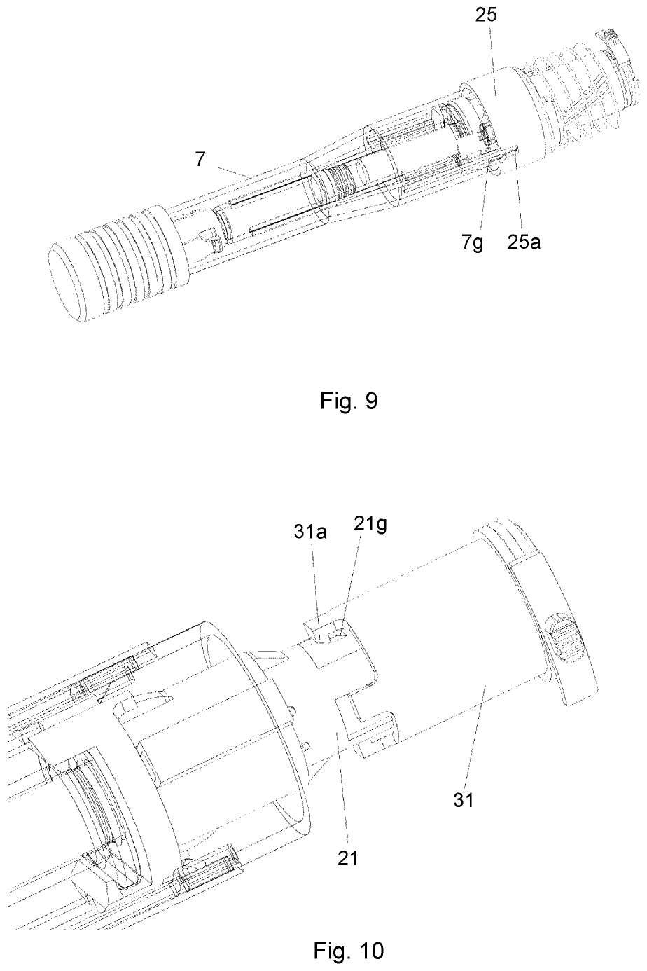

FIG. 9 shows the cassette front module 3 connected to the rear module 5 with the housing 17 removed.

The cassette front module 3 is configured to be moved axially relative to the housing 17. The cassette front module 3 is configured to move from an extended position to a retracted position to trigger discharging of medicament from the syringe 9. The cassette front module 3 is biased towards the extended position.

The interface sleeve 25 bears against a distal edge of the outer cover 7. The outer cover 7 is biased in an axial direction away from the distal housing end 17b by the resilient member 29.

The outer over 7 has an external axial rib 7g and the interface sleeve 25 comprises an axial recess 25a configured to engage with the external axial rib 7g to prevent rotation between the interface sleeve 25 and the outer cover 7.

FIG. 10 shows the release switch sleeve 31 with its anchoring hooks 31a engaging with a respective one of the protrusions 21g of the first sleeve member 21.

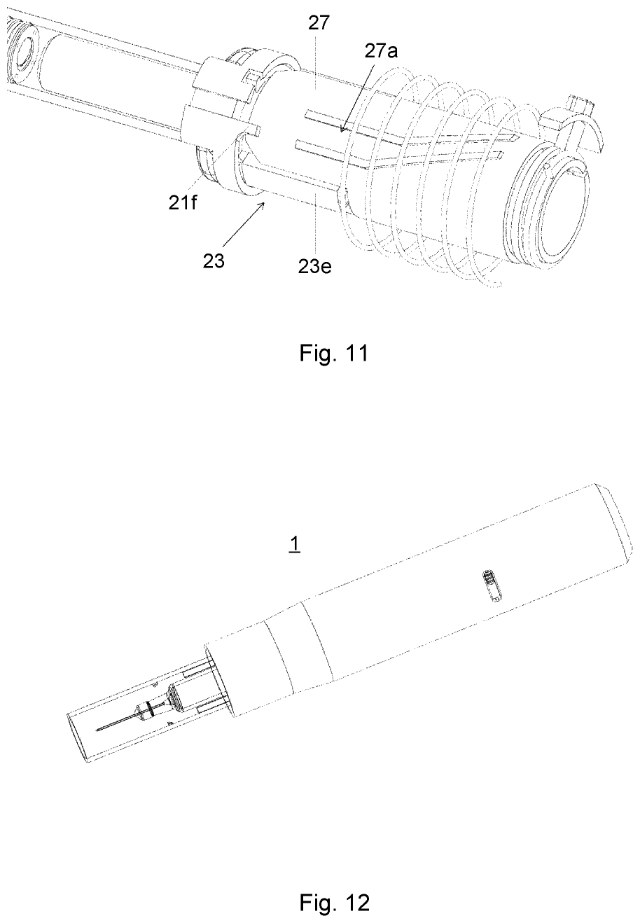

FIG. 11 depicts the rotator 27 arranged around the second sleeve member 23. The rotator 27 presses the tab 23c of the second sleeve member 23 radially inwards as the rotator 27 is slid over the second sleeve member 23 when the cassette front module 3 is connected to the rear module 5. The tab 23c is thus pressed radially inwards as long as the rotator 27 is arranged outside the second sleeve member 23. Since the tab 23c is located radially below the stop 21f, under the rotator 27, the second sleeve member 23 is able to rotate in the second direction.

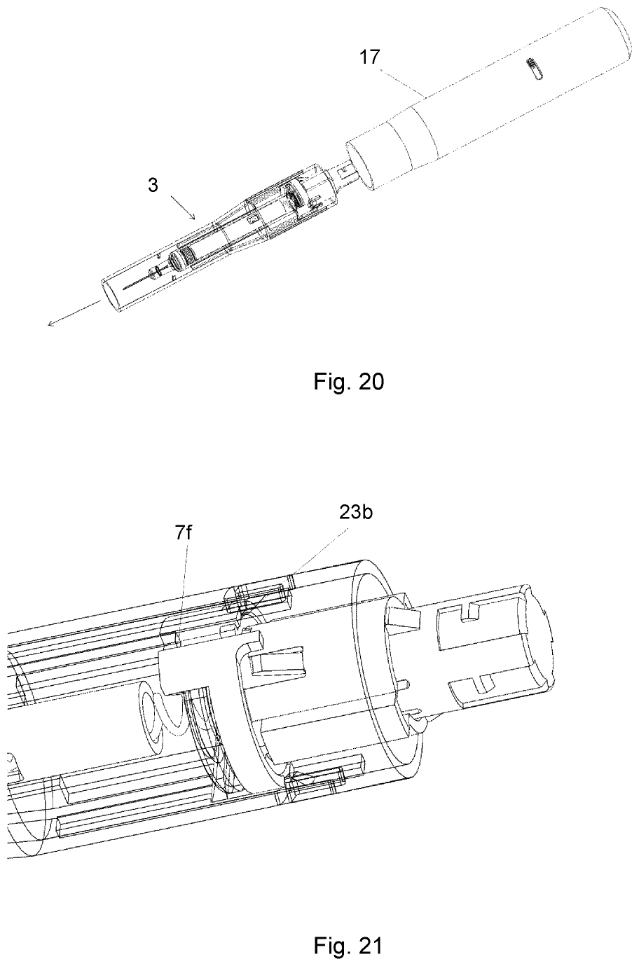

The operation of the medicament delivery device 1 will now be described with reference to FIGS. 12-21.

FIG. 12 shows the medicament delivery device 1 in a state ready to be used. Before this state is reached, the cassette front module 3 and the rear module 5 are attached to each other. This is done by inserting the cassette front module 3 into the proximal housing opening 17c of the rear module 5 and pushing the cassette front module 3 until the anchoring hooks 31a engage with a respective one of the protrusions 21g of the first sleeve member 21.

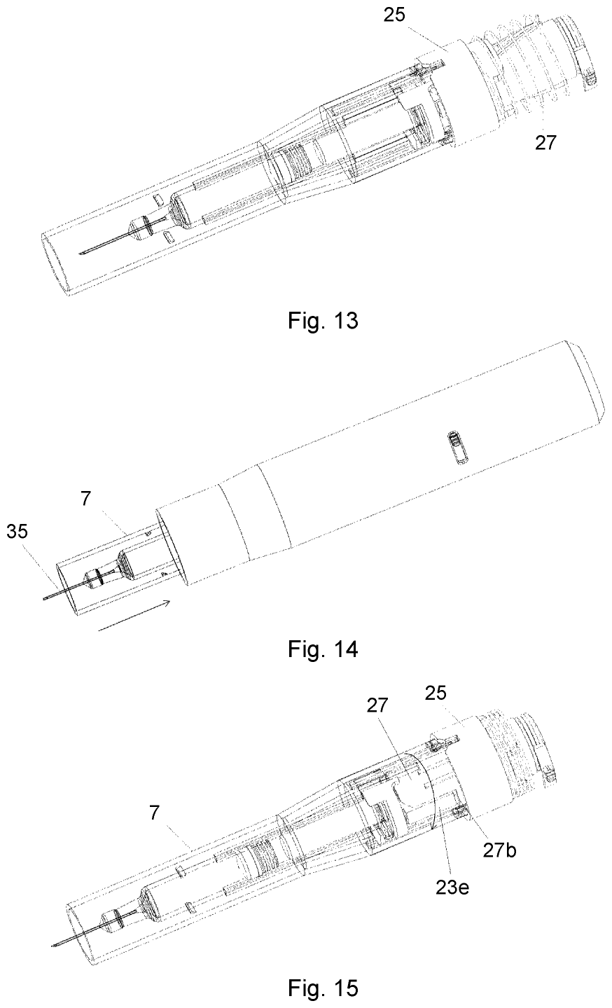

FIG. 13 shows the medicament delivery device in the state depicted in FIG. 12 without the housing 17. The interface sleeve 25 engages with the rotator 27.

FIG. 14 depicts the medicament delivery device 1 during medicament delivery. The medicament delivery device 1 is in this case placed against the site of injection and the user presses the housing 17 towards the skin. This causes the cassette front module 3 to move from the extended position towards the retracted position. The delivery member, in this example a needle 35, of the syringe, is thus exposed and penetrates the skin.

FIG. 15 shows the internal components of the medicament delivery device 1 in the state depicted in FIG. 14. The interface sleeve 25 has been moved axially inside the housing 17 by the movement of the cassette front module 3 from the extended position to the retracted position. This causes the rotator 27 to rotate.

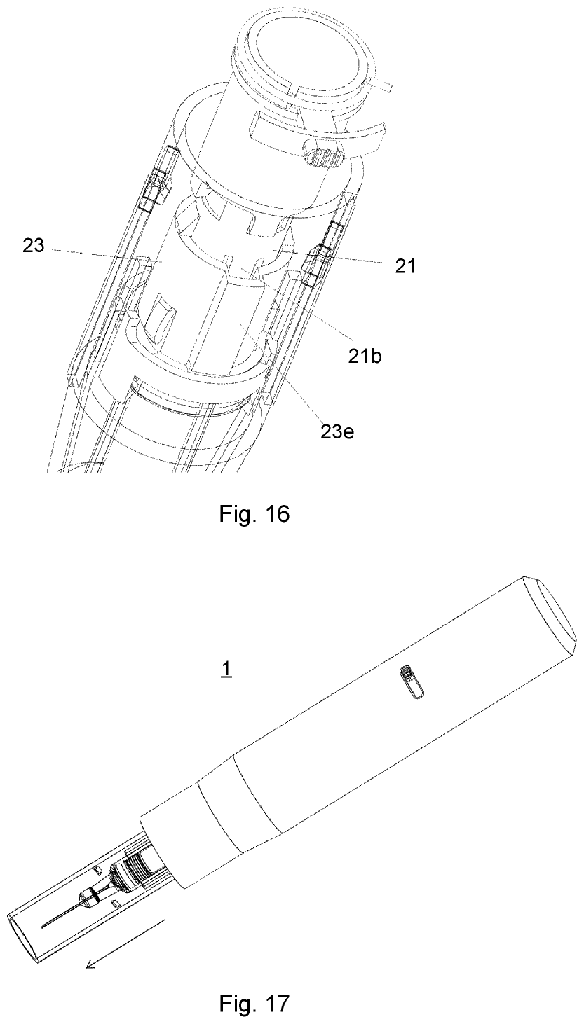

The rotator 27 has an axial cut-out 27b which receives and engages with the axial outer ribs 23e of the second sleeve member 23. As the rotator 27 is rotated in the second direction, the rotator 27 brings with it the second sleeve member 23 so that the second sleeve member 23 is also rotated in the second direction concurrently with the rotator 27. The sleeve recesses 23d eventually align with the flexible arms 21b which therefore are able to flex radially outwards from the plunger rod recesses 13b. This can be seen in FIG. 16, where the rotator 27 has been hidden to show the interaction between the first and second sleeve member 21 and 23. The plunger rod 13 is as a result released from its initial axial position and is thus able to discharge medicament from the syringe 9.

FIG. 17 shows the medicament delivery device 1 after medicament injection, when the medicament delivery device 1 has been removed from the site of injection. The cassette front module 3 thus returns to the extended position towards which it is biased by the resilient member

When the cassette front module 3 is returned to the extended position, the rotator 27 is rotated back by the linear movement of the interface sleeve 25 which returns to its initial axial position as the cassette front module 3 returns to the extended position. There is a play between the axial cut-out 27b and the axial outer ribs 23e in the circumferential direction which is designed so that the rotator 27 does not bring with it the second sleeve member 23. The second sleeve member 23 therefore maintains the release rotational position.

The cassette front module 3 is locked in the extended position after medicament injection. The cassette front module 3 can thus not be moved back into the housing 17 to the retracted position. To this end, the axial slot 21h, shown in FIG. 7, of the first sleeve member 21 has become axially aligned with the hook 23b at this stage. The hook 23b is arranged distally relative to the axial slot 21h. The axial slot 21h is also aligned with the axial rib 7f on the inner surface of the outer cover 7. The axial rib 7f is arranged proximally of the axial slot 21h. Thus, the hook 23b restricts movement of the axial rib 7f and thus of the outer cover 7 in the distal direction towards the distal housing end 17b.

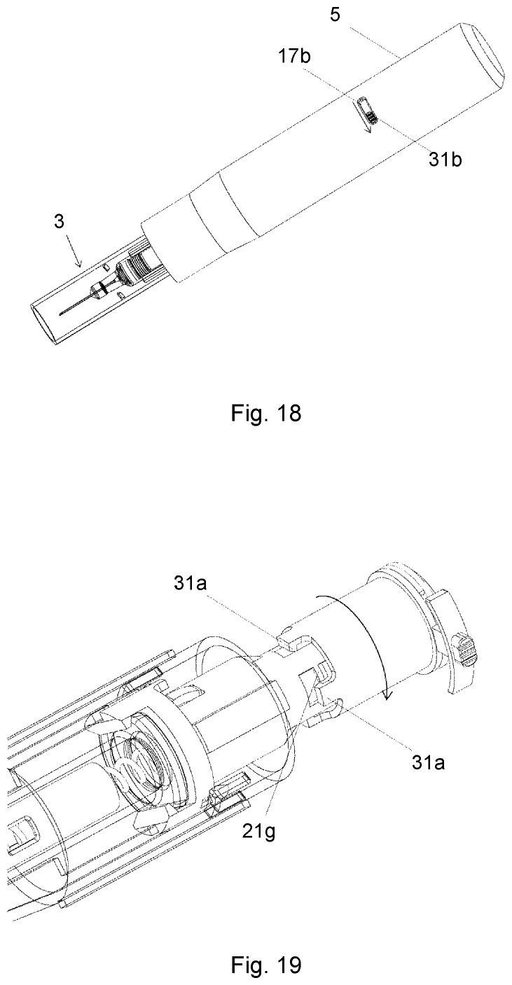

FIG. 18 shows when the cassette front module 3 is disconnected or detached from the rear module 5. The release switch 31b is moved by the user in the slot 17b in the housing 17 from the first position to the second. As a result, the release switch sleeve 31 disengages from the first sleeve member 21, as shown in FIG. 19. The cassette front module 3 can then be pulled out from the housing 17 of the rear module 5 and be disposed of. The rear module 5 is reusable and can thus be used with another cassette front module 3 by inserting the new cassette front module 3 into the housing 17.

FIG. 21 shows the cassette front module 3 after it has been removed from the rear module 5, in a locked-out state with the hook 23b arranged aligned with the axial rib 7f to prevent further use of the cassette front module 3.

The inventive concept has mainly been described above with reference to a few examples. However, as is readily appreciated by a person skilled in the art, other embodiments than the ones disclosed above are equally possible within the scope of the inventive concept, as defined by the appended claims.

1-13 (canceled)

14. A disposable cassette front module for a medicament delivery device comprising a reusable rear module which forms a rear portion of the medicament delivery device and to which the cassette front module is configured to be detachably connected to form a front portion of the medicament delivery device, the cassette front module comprising:
an outer cover configured to accommodate a syringe, the outer cover having a proximal outer cover end and a distal outer cover end; and
a power pack assembly comprising a plunger rod and a drive device configured to drive the plunger rod from an initial axial position relative to the outer cover towards the proximal outer cover end.

15. The cassette front module of claim 14, wherein the drive device is a drive spring, and wherein the cassette front module comprises a first sleeve member arranged around the plunger rod and configured to engage with the plunger rod to hold the plunger rod in the initial axial position.

16. The cassette front module of claim 15, wherein the first sleeve member has a distal end provided with a proximal radial surface and the drive spring has a distal drive spring end bearing against the proximal radial surface.

17. The cassette front module of claim 15, wherein the plunger rod has a hollow interior configured to receive the drive spring, wherein the plunger rod has a distal radial surface, and the drive spring has a proximal drive spring end bearing against the distal radial surface.

18. The cassette front module of claim 14, wherein the plunger rod has plunger rod recesses and the first sleeve member has radially flexible arms configured to engage with a respective one of the plunger rod recesses to hold the plunger rod in the initial axial position, wherein the cassette front module comprises a second sleeve member arranged concentrically with and radially outside the first sleeve member, the second sleeve member having an inner surface with sleeve recesses configured to receive a respective one of the radially flexible arms when the second sleeve member is in a release rotational position relative to the plunger rod, wherein the second sleeve member is configured to be rotationally locked relative to the first sleeve member in an initial rotational position to prevent the first sleeve recesses to move to the release rotational position.

19. The cassette front module of claim 18, wherein the second sleeve member comprises a radially flexible circumferentially extending arm provided with a hook and the first sleeve member has a radial opening configured to receive the hook to prevent the second sleeve member to rotate in a first direction but to enable the second sleeve member to rotate in a second direction.

20. The cassette front module of claim 18, wherein the second sleeve member comprises a radially flexible axial tab extending radially outwards with an increasing amount in an axial direction towards the proximal outer cover end, and the first sleeve member has a stop extending axially towards the distal outer cover end adjacent to the tab, the stop being configured to prevent the second sleeve member to rotate in a second direction.

21. The cassette front module of claim 20, wherein the outer cover has an inner surface provided with an axial rib arranged axially and radially aligned with the tab, the axial rib having a distal end face closer to the proximal outer cover end than the tab, the tab thereby restricting axial movement of the outer cover relative to the second sleeve member in a direction towards the tab.

22. The cassette front module of claim 14, comprising a cap configured to be attached to the outer cover, wherein the proximal outer cover end has a proximal opening and the cap comprises flexible axial arms configured to extend into the outer cover through the proximal opening, wherein the outer cover has an inner surface provided with radially inwards extending protrusions configured to bear against a respective one of the flexible axial arms at an axial position of the outer cover where, in use, a syringe is arranged, the protrusions being configured to prevent the flexible axial arms to move in an axial direction towards the proximal outer cover end and the syringe restricting radial inwards flexing of the flexible axial arms.

23. A medicament delivery device comprising:
a reusable rear module comprising a housing having a proximal housing end and a distal housing end, the proximal housing end having a proximal housing opening, and
the cassette front module of claim 14, wherein the outer cover is configured to be received by the proximal housing opening, the cassette front module being configured to be moved from an extended position relative to the housing to a retracted position, wherein the rear module comprises a resilient member configured to bias the cassette front module towards the extended position.

24. The medicament delivery device of claim 23, wherein the rear module comprises:
an interface sleeve biased towards the proximal housing end, the interface sleeve bearing against a distal edge of the outer cover, wherein the outer cover has an external axial rib, and the interface sleeve comprises an axial recess configured to engage with the external axial rib to prevent rotation of the interface sleeve relative to the outer cover, and
a rotator provided with a guide structure, the rotator being rotationally engaged with the second sleeve member and being configured to press the tab radially inwards to enable rotation of the second sleeve member in the second direction,
wherein the interface sleeve is configured to cooperate with the guide structure when the cassette front module is moved from the extended position towards the retracted position causing the rotator and the second sleeve member to rotate in the second direction.

25. The medicament delivery device of claim 23, wherein the rear module comprises a release switch sleeve which is torsionally biased and axially locked relative to the housing, the release switch sleeve comprising a plurality of anchoring hooks and the first sleeve member comprises a plurality of protrusions configured to engage with a respective one of the anchoring hooks to prevent the rear module from disengaging from the cassette front module.

26. The medicament delivery device of claim 25, wherein the release switch sleeve comprises a release switch extending through a circumferentially extending slot of the housing, wherein the release switch is configured to be slid in the slot from a first position towards which it is torsionally biased to a second position to disengage the anchoring hooks from the protrusions and thereby disengage the rear module from the cassette front module.
